(19) United States
(12) Patent Application Publication
(10) Pub. No.: US 2020/0325435 A1
(43) Pub. Date: Oct. 15, 2020

(54) APPARATUS AND METHODS FOR MEASURING, MONITORING, AND CONTROLLING CHEMICAL CONCENTRATIONS OF ACTIVE INGREDIENTS IN PLANT AND PLANT CROP TREATMENT SOLUTIONS

(71) Applicant: AGROFRESH INC., Philadelphia, PA (US)

(72) Inventors: Andrés LÓPEZ GÓMEZ, Valencia (ES); José M. PÉREZ CHECA, Valencia (ES)

(21) Appl. No.: 16/848,139

(22) Filed: Apr. 14, 2020

(30) Foreign Application Priority Data
Apr. 15, 2019 (ES) 201930341

(51) Int. Cl. C12M 1/34; G01N 33/18; G01N 27/27

(52) U.S. Cl. CPC C12M 41/32; G01N 33/1826; G01N 2033/184; G01N 27/27; C12M 41/12

(57) ABSTRACT

The present disclosure relates to an apparatus and methods for measuring, monitoring, and/or controlling the chemical concentration of one or more active ingredients in a solution for treating plants and plant crops, such as fruits and vegetables.

### CROSS-REFERENCE TO RELATED APPLICATION

This application claims priority to Spanish Patent Application No. 201930341 filed Apr. 15, 2019 (04.15.2019), the entire disclosure of which is incorporated by reference herein in its entirety.

### FIELD OF THE PRESENT DISCLOSURE

The present disclosure relates to an apparatus and methods for measuring, monitoring, and/or controlling the chemical concentration of one or more active ingredients in a solution for treating plants and plant crops, such as fruits and vegetables.

### BACKGROUND

Traditional pre-harvest and post-harvest treatment solutions often comprise crop protection compounds comprising active ingredients or chemicals that prolong the shelf life and/or prevent degradation of plant crops, such as fruit and/or vegetable crops. Active ingredients in standard pre-harvest and post-harvest plant crop treatment solutions often include, but are not limited to chemicals, fungicides, sanitizers, pesticides, benzoxaborole derivatives, ripening inhibitor agents (e.g., 1-methylcyclopropene), and combinations thereof. In particular, post-harvest crop protection compounds or chemicals, such as pesticides and fungicides are often applied to plant crops using spraying, dipping, and drenching methods that occur in large treatment tanks.

However, spraying, dipping, and drenching application techniques are known to encounter problems when maintaining a specific concentration of active ingredient in the treatment solution and/or uniform distribution of active ingredient onto treated plant crops. For example, set concentrations of a pesticide or fungicide treatment may rise too high if their concentration is not properly monitored resulting in concentrations that exceed regulatory maximum residue limits. Alternatively, set concentrations of a pesticide or fungicide treatments may fall below the minimum level required for efficacy.

Tools currently available to monitor and control the concentration of active ingredients present in crop protection treatment solutions are limited to methods and technologies that require significant amounts of time to obtain an accurate reading, such as high-performance liquid chromatography (HPLC) and/or gas chromatography (GC). Often, by the time that results regarding the concentration of the active ingredients are measured, analyzed and/or determined, the concentration of the active ingredient comprised in the crop protection treatment solution may have changed. On the other hand, current systems that are capable of providing a fast measurement of the concentration of active ingredients in a crop protection treatment solution do not reliably produce accuracy.

The present disclosure relates to an apparatus and methods to address the challenges presented by the current technology. Thus, the apparatus and methods of the present disclosure are related to measuring, monitoring, and/or controlling one or more active ingredients in treatment solutions applied to plants and plant crops, such as fruits and vegetables. More specifically, the apparatus and methods of the present disclosure provide a fast, reliable, real-time, online system to assess, determine, and manipulate the concentration of a wide spectrum (i.e., a plurality) of chemical substances and/or active ingredients used for treatment of plants and plant crops, such as fruits and vegetables. Ultimately, the apparatus and methods described herein provide a new apparatus and treatment methods with technological benefits to preserve the freshness of plants and plant crops by delaying premature ripening or degradation, and therefore, extending shelf life of the treated plants and plant crops.

### SUMMARY OF THE INVENTION

The present disclosure provides a method of measuring and monitoring a concentration of one or more active ingredients in a treatment solution. This method of the present disclosure may comprise obtaining a treatment solution sample with an initial concentration of one or more active ingredients. The method may also comprise preparing the treatment solution sample for analyzing the concentration of the one or more active ingredients resulting in a modified treatment solution sample. In addition, the method may comprise performing analysis of the modified treatment solution sample in a measurement cell and generating an electric signal. Further, the method comprises analyzing the electric signal to identify the initial concentration of the one or more active ingredients comprised in the modified treatment solution sample.

The present method may be performed in real-time, continuously, or on-demand. The present method further comprises use of a calibration curve to analyze the electric signal to identify the concentration of the one or more active ingredients. In one embodiment, the one or more active ingredients of the present method may be selected from the group consisting of pesticides, insecticides, fungicides, herbicides, food additives, processing aids, sanitizing agents, benzoxaborole derivatives, and ethylene inhibitors.

In another embodiment, the one or more active ingredients are a fungicide. In a further embodiment, the fungicide is selected from the group consisting of imazalil, thiabendazole, fludioxonil, prochloraz, propioconazole, pyrimethanil, orthophenylphenol, azoxystrobin, difenoconazole, and diphenylamine In yet a further embodiment, the one or more active ingredients are a sanitizing agent, wherein the sanitizing agent. In another embodiment, the sanitizing agent is peracetic acid, acetic acid, ethanol, hydrogen peroxide, chlorine, hypochlorous acid, sodium hypochlorites, calcium hypochlorites, or combinations thereof.

The present disclosure is also directed to a method of controlling a desired concentration of one or more active ingredients in a treatment solution. The method comprises measuring the initial concentration of one or more active ingredients according to the present method of measuring. The method further comprises assessing the measured initial concentration of the one or more active ingredients in the treatment solution to determine when it is outside the acceptable threshold of deviation from the desired concentration of the one or more active ingredients. In addition, the method comprises delivering a carrier or an additional concentration of the one or more active ingredients to the treatment solution to correct any deviation from the acceptable threshold. Further, the method comprises performing a subsequent measurement of the concentration of the one or more active ingredients in the treatment solution to confirm that it remains at or about the desired concentration of the one or more active ingredients or within the threshold. Importantly, the delivering may be performed manually or automatically.

Finally, the present disclosure is directed to an apparatus or an integrated system. The present apparatus automatically measures, monitors, and optionally controls the concentration of one or more active ingredients in a treatment solution. The apparatus may further comprise, consist essentially of, or consist of one or more of the following components: a) an automated sample preparation system, b) an automated system for analyzing the sample, c) a signal analysis system, d) a control system, and e) an automated delivery system.

In an illustrative embodiment, the apparatus comprises a control system, wherein the control system comprises a plurality of recorded measurements of the one or more active ingredient concentrations. In a different embodiment, the apparatus comprises an automated delivery system. In another embodiment, the apparatus comprises a control system and an automated delivery system. In a further embodiment, the control system optionally communicates with the automated delivery system to perform delivery of the corresponding amount of the one or more active ingredients or a carrier when the measured concentration is outside the acceptable threshold of deviation from the desired concentration of the one or more active ingredients. Finally, the apparatus of the present method may comprise one or more sensors selected from the group consisting of thermometers, hygrometers, and flow meters.

### BRIEF DESCRIPTION OF THE DRAWINGS

A brief description of the drawings is as follows.

FIG. 1A is a schematic that denotes the various components of one embodiment of the present apparatus and methods.

FIG. 1B is a schematic of one embodiment of a system for delivering reactants to the one or more measurement cell(s) of the present apparatus for sample preparation.

FIG. 1C is a schematic of one embodiment of a pump of the present apparatus.

FIG. 1D is a schematic of one embodiment of a valve of the present apparatus.

FIG. 1E is a schematic of one embodiment of a column of the present apparatus.

FIG. 1F is a schematic of one embodiment of the measurement cell(s) and light source of the present apparatus.

FIG. 1G is a schematic of one embodiment of an optical filter of the present apparatus.

FIG. 1H is a schematic of one embodiment of a photo sensor of the present apparatus.

FIG. 1I is a schematic of how one of the sensor systems of the present apparatus detects the signal correlated to the concentration of active ingredients in the modified treatment solution sample.

FIG. 2A is a flow chart of one embodiment of the method of measuring a concentration of one or more active ingredients of the present disclosure.

FIG. 2B is a flow chart of one embodiment of the method of delivering a concentration of one or more active ingredients of the present disclosure.

FIG. 3 is a chromatograph that shows detection of multiple different fungicides in a single treatment solution.

FIG. 4A is a chromatograph that shows detection of fludioxonil in a treatment solution.

FIG. 4B is a chromatograph that shows detection of imazalil in a treatment solution.

FIG. 4C is a chromatograph that shows detection of orthophenylphenol in a treatment solution.

FIG. 4D is a chromatograph that shows detection of prochloraz in a treatment solution.

FIG. 4E is a chromatograph that shows detection of propioconazol in a treatment solution.

FIG. 4F is a chromatograph that shows detection of pyrimethanil in a treatment solution.

FIG. 4G is a chromatograph that shows detection of thiabendazole in a treatment solution.

FIG. 5A is a schematic of one embodiment of a chemical reaction that enables peracetic acid (PAA) to generate a signal that is detected by the present methods and apparatus.

FIG. 5B is a graph that demonstrates use of a calibration curve to correlate the signal generated by an active ingredient, such as peracetic acid (PAA), to its concentration.

FIG. 6 is a flow chart of an embodiment of the methods of measuring, monitoring, delivering, and controlling a set point concentration of one or more active ingredients of the present disclosure.

### DETAILED DESCRIPTION

### Definitions

As used herein, the terms “administration” and “administering” refer to the act of giving a drug, compound, pesticide, fungicide, or other agent to a plant, plant crop, and/or plant parts in vivo or in vitro.

The terms “analog” and “derivative” are interchangeable, and refer to a natural or man-made modification of at least one position of a given molecule or compound. For example, a derivative of a given compound or molecule may be modified either by addition of a functional group or atom, removal of a functional group or atom or change of a functional group or atom to a different functional group or atom (including, but not limited to, isotopes).

The term “automated” refers to a process or task that is independently operated by one or more instrument, equipment, and/or apparatus by following a set of sequential steps. The term “automatically” also refers to such process or task at least requiring about 90% of the action to be performed by these elements. The term “semi-automatically” refers to such process or task at least requiring about 50% of the action to be performed by these elements.

The term “carrier” refers to a material, composition, or medium, such as a liquid or solid filler, diluent, excipient, solvent, gas, or encapsulating material, involved in carrying or delivering an active ingredient, compound, analog, or derivative to the intended target or location. Alternatively, carriers of the present disclosure may comprise, consist essentially, of or consist of no active ingredient at all. A carrier must be acceptable in the sense of being compatible with the other ingredients of the formulation and not injurious to the plant or plant parts.

A “combination” as used herein refers to a joining of one or more components to another or a plurality of components. The combination may comprise, consist essentially of, or consist of atoms, compounds, compositions, components, constituents, elements, moieties, molecules, or mixtures. A combination includes, but is not limited to, a mixture.

The term “comprising” refers to a composition, compound, formulation, or method that is inclusive and does not exclude additional elements or method steps. The term “comprising” also refers to a composition, compound, formulation, or method embodiments of the present disclosure that is inclusive and does not exclude additional elements or method steps.

The term “consisting of” refers to a compound, composition, formulation, or method that excludes the presence of any additional component or method steps. The term “consisting of” also refers to a compound, composition, formulation, or method of the present disclosure that excludes the presence of any additional component or method steps.

The term “consisting essentially of” refers to a composition, compound, formulation or method that is inclusive of additional elements or method steps that do not materially affect the characteristic(s) of the composition, compound, formulation or method. The term “consisting essentially of” also refers to a composition, compound, formulation or method of the present disclosure that is inclusive of additional elements or method steps that do not materially affect the characteristic(s) of the composition, compound, formulation, or method.

The term “compound(s)” refers to any one or more chemical entity, moiety, pharmaceutical, drug, active agents, chemicals, and the like that can be used to treat, diagnose, or prevent a disease, disorder, infection of plants, plant parts, and/or plant crops. A compound can be determined to be effective or efficacious by using the screening or experimental methods of the present application.

When a compound is described herein as being present as a gas in an atmosphere, the rate that the compound is applied is reported as milligram (mg) of active ingredient (i.e., the compound) per liter (L) of container or chamber headspace volume.

The terms “composition” or “compositions” refer to the combination of one or more compounds with or without another agent, making the composition especially suitable for use in treating plants, plant crops, and plant parts.

The term “component” refers to a constituent part of a compound or a composition. For example, components of a composition can include a compound, a carrier, and any other agent present in the composition.

The term “continuous” refers to performing an action uninterruptedly, such as measuring, monitoring and/or controlling without interruption for extended periods such as hours, days or months.

The terms “effective” and “effective amount” refer to the amount of a composition or compound sufficient to effect beneficial or desired results. An effective amount can be administered in one or more applications or dosages and is not intended to be limited to a particular formulation or administration route.

Exemplary embodiments of the compounds of the present disclosure comprise pesticides, insecticides, fungicides, herbicides, food additives, processing aids, sanitizing agents, benzoxaborole derivatives and ethylene inhibitors. Illustrative pesticides and/or fungicides of the present disclosure comprise, consist essentially of, or consist of imazalil, thiabendazole, fludioxonil, prochloraz, propioconazole, pyrimethanil, orthophenylphenol, azoxystrobin, difenoconazole, and diphenylamine, which may encompass diastereomers and enantiomers of the illustrative compounds. Enantiomers are defined as one of a pair of molecular entities which are mirror images of each other and non-superimposable. Diastereomers or diastereoisomers are defined as stereoisomers other than enantiomers. Diastereomers or diastereoisomers are stereoisomers not related as mirror images. Diastereoisomers are characterized by differences in physical properties.

The terms “inhibit,” “inhibitory,” or “antagonistic” refers to the property of a compound that decreases, limits, inhibits, or blocks the action or function of an infection or another compound.

The term “isolated” refers to the separation of a material from at least one other material in a mixture or from materials that are naturally associated with the material. For example, a compound synthesized synthetically is separated from a starting material or an intermediate.

The term “on-demand” refers to performing a process or task when requested or needed, such as a measurement or an analysis.

“Optional” or “optionally” refers to a circumstance in which the subsequently described event or circumstance may or may not occur, and that the description includes instances where said event or circumstance occurs and instances in which it does not. “Optionally” is inclusive of embodiments in which the described condition is present and embodiments in which the described condition is not present.

The term “plant(s)” and “plant parts” include, but not limited to, plant cells and plant tissues, such as leaves, calli, stems, roots, flowers, pollen, and seeds. A class of plants that may be used in the present disclosure is generally as broad as the class of higher and lower plants including, but not limited to, dicotyledonous plants, monocotyledonous plants, and all agronomic crops.

“Agronomic crops” and/or “plant crops” of the present disclosure include, but are not limited to, horticultural crops. Horticultural crops include, but are not limited to, vegetable crops, fruit crops, edible nuts, flowers and ornamental crops, nursery crops, aromatic crops, and medicinal crops. More specifically, horticultural crops of the present disclosure include, but are not limited to, fruits and vegetables of any kind with no limitations.

The terms “plant material” or “plant part” include, but are not limited to, leaves, calli, stems, roots, flowers or flower parts, fruits, pollen, egg cells, zygotes, seeds, cuttings, cell or tissue cultures, or any other part or product of a plant. In an embodiment, plant material or plant part includes cotyledon and leaf. In an embodiment, plant material or plant part includes root tissues and other plant tissues located underground.

The terms “purified” or “substantially purified” refers to the removal of inactive or inhibitory components (e.g., contaminants) from a composition to the extent that 10% or less (e.g., 10% or less, 9% or less, 8% or less, 7% or less, 6% or less, 5% or less, 4% or less, 3% or less, 2% or less, or 1% or less) of the composition comprises inactive components, compounds, or carriers.

The term “real-time” refers to a characteristic of a device to communicate data or information immediately or within a very short time after obtaining the information.

The term “sensor” refers to a device that detects physical, chemical or biological events and changes in its environment by responding in situ to these inputs or by generating a signal that is transmitted to an electronic device where it is interpreted into useful data or information. The term “sensing system” may be applied when the sensor is part of a system comprising of other elements.

The terms “treating,” “treat,” or “treatment” refer to an application or administration of a compound or agent into, onto, or throughout a plant, plant crop, and/or plant part where the objective is to slow down (e.g., lessen or postpone) the onset of an undesired physiological or biological condition (e.g., ripening, decay, and/or degradation), to reduce symptoms of an infection, condition, or disorder, or to obtain beneficial or desired results, such as partial or total restoration or inhibition in decline of a parameter, value, function, metric, or result that had or would become abnormal. Beneficial or desired results include, but are not limited to, alleviation of undesired effects; diminishment of the extent or vigor or rate of development of the infection, condition, or disorder; stabilization (i.e., not worsening) of the state of the condition, infection or disorder; delay in onset or slowing of the progression of the infection, condition, or disorder; amelioration of the infection, condition, or disorder; and enhancement or improvement of the infection, condition, or disorder.

### Compounds and Components of the Present Methods

The present disclosure is directed to an apparatus and methods of monitoring, measuring, and controlling compositions comprising chemical compositions and/or active ingredients that improve the shelf life and/or reduce, prevent, and/or delay degradation of plants and plant crops, such as fruit and/or vegetable crops. Active ingredients of the present methods may comprise, consist essentially of, or consist of any compound or composition that is effective to improve the shelf life and/or reduce, prevent, and/or delay degradation of plants and plant crops, such as fruit and/or vegetable crops.

Any effective combination of different types, kinds, and classes of active ingredients may be used individually or as a mixture or combination in the present methods and apparatus. Illustrative active ingredients of the present disclosure comprise, consist essentially of, or consist of pesticides, insecticides, fungicides, herbicides, food additives, processing aids, sanitizing agents, benzoxaborole derivatives, ethylene inhibitors, and/or combinations thereof.

An exemplary active ingredient of the present methods and apparatus is a fungicide. While any fungicide may be comprised in the present method and/or apparatus, exemplary fungicides of the present disclosure comprise, consist essentially of, or consist of imazalil, thiabendazole, fludioxonil, prochloraz, propioconazole, pyrimethanil, orthophenylphenol, azoxystrobin, difenoconazole, diphenylamine, and analogs or derivatives thereof. In addition, any effective combination of fungicides may be comprised in the methods and apparatus of the present disclosure.

Another illustrative example of the active ingredient of the present disclosure comprises, consists essentially of, or consists of a sanitizing agent. A sanitizing agent of the present disclosure comprises, consists essentially of, or consists of peracetic acid. Other sanitizing agents of the present disclosure comprise, consist essentially of, or consist of acetic acid, ethanol, hydrogen peroxide, chlorine, hypochlorous acid, sodium hypochlorites, calcium hypochlorites, or combinations thereof. Any effective combination of sanitizing agents may be comprised in the methods and apparatus of the present disclosure.

Active ingredients of the present disclosure may be administered and/or applied to plants and plant crops to effectively extend shelf life and/or reduce, delay, or prevent degradation. Active ingredients may be used in any form, including, but not limited to, a liquid, a solid (e.g., a powder), or a gaseous composition. In particular, the present method provides application of the active ingredients or chemical compounds as a spray, a mist, a gel, a thermal and non-thermal fog, a dip, a drench, a vapor, or a gas.

An exemplary application method comprises administering the one or more active ingredients comprised within a treatment solution by spraying, dipping, and/or drenching the treatment solution onto plants and plant crops, such as fruit or vegetable crops. Additional examples of treatment administration of the one or more active ingredients of the present disclosure may include, but are not limited to, release from a sachet, a synthetic or natural film, a liner or other packaging materials, a gas-releasing generator, compressed or non-compressed gas cylinder, a droplet inside a box, or other similar methods.

As described herein, treatment solutions of the present disclosure may comprise, consist essentially of, or consist of one or more active ingredients of the present disclosure to provide protection to plants and plant crops from premature ripening and from fungal and bacterial attack to extend shelf-life when administered, applied, or exposed to the plants and plant crops. Treatment solutions of the present disclosure may also comprise, consist essentially of, or consist of one or more active ingredients and a carrier. The treatment solution of the present disclosure typically comprises, consists essentially of, or consists of about 0.00001% to about 10% of the total amount or concentration of the one or more active ingredients. The total amount or concentration of the one or more active ingredients of the present disclosure comprises, consists essentially of, or consists of the sum of the amount or concentration of each of the one or more active ingredient in the treatment solution. Therefore, the treatment solution of the present disclosure generally comprises, consists essentially of, or consists of about 90% to about 99.99999% of the carrier.

Carriers, also called treatment carriers, of the present disclosure may be combined with any active ingredient or chemical compounds to form a treatment solution. Carriers of the present disclosure may comprise liquids, gases, oils, solutions, solvents, chemicals, and/or combinations thereof. For example, an exemplary liquid carrier of the present disclosure may comprise water, buffer, saline solution, a solvent, and combinations thereof.

In addition to the carriers, other components may be comprised in the treatment of the present disclosure including, but not limited to adjuvants, surfactants, excipients, dispersants, emulsifiers, and combinations thereof. Such other components may be comprised in the treatment solution, along with the one or more active ingredients and the carrier in order to better effectuate the active ingredient on the plants and plant crops. Typically, the treatment solution may comprise additional components in the range of about 0.01% to about 5%

Treatment solutions may be applied and/or administered to plants and plant crops by any methods known or used in the art, including, but not limited to spraying, dipping, drenching, fogging, etc. In an illustrative embodiment, treatment solution comprising the one or more active ingredients or compounds of the present disclosure may be applied to plants and plant crops while the plants and plant crops are located within a tank or a chamber.

A tank or a chamber of the present disclosure may be any container sufficient to hold plants and plant crops and effectively administer a treatment solution comprising the one or more active ingredients of the present disclosure. The tank or chamber of the present disclosure may be sealable or non-sealable. An illustrative tank or chamber of the present disclosure is sealable.

In addition, the tank or chamber of the present disclosure may be permeable such that air and gases are able to permeate through or in and out of the tank. Alternatively, the tank or chamber of the present disclosure may be impermeable such that air and gases are not able to permeate through or in and out of the tank. In addition, a tank or a chamber of the present disclosure may be made of plastic, glass, metal, or any other semipermeable or impermeable material that enables effective administration of the active ingredient and/or the treatment solution described herein to plants and plant crops.

The tank or chamber of the present disclosure may be of any size that is large enough to hold plants and plant crops to be treated with the treatment solution and/or active ingredients of the present disclosure. In an exemplary embodiment, the tank or chamber of the present disclosure may comprise, consist essentially of, or consist of a plurality of plants and plant crops to be treated. For example, an exemplary tank or chamber may have a volume or capacity of about 20 to about 80000 liters (L), from about 150 L to about 47500 L, from about 300 L to about 45000 L, from about 500 L to about 32500 L, from about 750 L to about 41000 L, from about 800 L to about 40000 L, from about 850 L to about 30000 L, from about 1500 L to about 80000 L, from about 10000 L to about 80000 L, and from about 1200 L to about 25000 L.

Liquid treatment solutions of the present disclosure may be applied to the plants and plant crops within and outside of a tank or a chamber. In an embodiment where the treatment solutions are applied to plants and plant crops within a tank or a chamber, the rate of application may be reported as the percent (%) of active ingredient per liter of treatment solution. For example, the rate that the active ingredient in the treatment solution may be effectively applied to plants and plant crops, such as fruits or vegetables, may range from 0.00001% to 10% of active ingredient per liter of treatment solution.

More specifically, the rate of applying the treatment solution to plants and plant crops may be from about 0.0001% to about 1%, from 0.001% to about 0.1%, from about 0.005% to about 0.05%, and from 0.01% to about 0.05% of active ingredient per liter of treatment solution.

Any plants, plant parts, and plant crops may be treated using the present methods and apparatus. A class of plants that may be treated in the present disclosure is generally as broad as agronomic crops. Agronomic crops of the present method include horticultural crops. Horticultural crops include, but are not limited to, vegetable crops, fruit crops, edible nuts, flowers and ornamental crops, nursery crops, aromatic crops, and medicinal crops. More specifically, any types of fruits and vegetables, without limitations, are illustrative plants and plant crops of the present methods and apparatus.

Plants and plant crops of the methods and apparatus of the present disclosure also include any agricultural crops in any production cycle. For example, plants and plant crops in greenhouse production, post-harvest during field packing, palletization, in-box, during storage, and throughout the distribution network are comprised within the present disclosure. Further, any plants and plant crops that may be held in a contained space may also be used in the present methods and apparatus. A contained space includes, but is not limited to, a cold-storage room, a marine container, an air container, a train car or local vehicle, a transport truck or trailer, a box or a pallet-wrap, a greenhouse, a grain silo or similar. In some embodiments, a contained space of the present disclosure may also serve as a tank or a chamber of the present disclosure.

### Apparatus or System of the Present Methods

The present disclosure is also related to an apparatus or an integrated system that measures, monitors, and/or controls the one or more active ingredients or chemical compounds that are being applied or administered to plants and plant crops comprised in a treatment tank or a treatment chamber. More specifically, the apparatus of the present disclosure is capable of measuring, monitoring, and controlling one or more active ingredients in the treatment solution of the present disclosure before, during, and after its application to a plurality of plants and plant crops in a treatment tank. In particular, the apparatus or system of the present disclosure allows and/or enables measuring, monitoring, and controlling of the amount or concentration of a wide spectrum and multiple number (e.g., a plurality) of different active ingredients or chemical compounds, such as fungicides, sanitizing agents, food additives, processing aids, and combinations thereof that are commonly used in pre- and post-harvest plant crop treatment processes that are often performed within tanks.

The apparatus or system of the present disclosure, including one or more components thereof, may be attached to, affixed to, and/or connected to a tank or a chamber of the present disclosure. In an illustrative embodiment, the apparatus or system of the present disclosure, including one or more components thereof, is connected to the tank or chamber or to one another via one or more connectors, including, but not limited to one or more cords, wires, and/or tubes, and combinations thereof. For example, the one or more connectors may be used to connect the present apparatus or system to the tank or chamber, such that a sample (e.g., of the treatment solution) may be transferred from the tank to the apparatus or from the apparatus to the tank via the one or more connectors.

The apparatus or system of the present disclosure, including one or more components thereof, may also be located on the interior or the exterior of the tank or chamber. In an illustrative embodiment, the apparatus or system of the present disclosure, including one or more components thereof, are attached to, affixed to, and/or connected to the interior of the tank or chamber while the apparatus or system is located on the exterior of the tank. In an exemplary embodiment, the apparatus or system of the present disclosure, including one or more components thereof, are attached to, affixed to, and/or connected to the inside of the tank or chamber while also being located within the interior of the tank or chamber.

The apparatus or system of the present disclosure may comprise one or more components. For example, the apparatus or system of the present disclosure may comprise multiple or a plurality of components. In particular, one or more components of the present apparatus or methods may comprise, consist essentially of, or consist of one or more of the following components or devices: a) an automated sample preparation system, b) an automated sample analysis system (i.e., a system for analyzing the sample), c) a signal analysis system, d) a control system, and e) an automated delivery system.

The automated sample preparation system of the present disclosure may comprise, consist essentially of, or consist of one or more of the following components or devices: a) an automatic sampler, b) a particle filtering system, and c) an automated reagent delivery system. The automated sampler may robotically obtain or comprise a sample of the treatment solution. The particle filtering system may be used to optionally filter and/or dilute the treatment solution sample in preparation for sample analysis.

The automated reagent delivery system may further comprise, consist essentially of, or consist of one or more pumps and/or syringes. For example in an illustrative embodiment, syringes are comprised in a dosing syringes system. The dosing syringes system of the present automated reagent delivery system is used to add the reagents for preparation of the treatment solution sample.

Preparation of the treatment solution sample for analysis by the automated sample preparation system of the present apparatus may comprise addition of one or more reactants or reagents to the sample. Effective amounts of reactants and/or reagents may be added to the sample simultaneously or consecutively. Typically, addition of the reagent or reactant to the treatment solution sample will generate a chemical, photometric, and/or enzymatic reaction that will modify the treatment solution sample to create and/or produce a modified sample, which is further analyzed by the automated system of the present apparatus.

Generally, the modified sample is then flushed, flowed, or pumped to and/or through additional components of the apparatus by a pump device. The pump of the apparatus may be a high-pressure pump device. For example, the pump may propel the modified sample to a column and/or a light sensing system (e.g., a detector) for sample analysis.

The apparatus of the present disclosure may also comprise a valve. The valve of the present apparatus may be an injection valve. The injection valve of the present apparatus may be a six-way valve. Notably, use of a six-way valve allows and/or enables the treatment solution sample to be introduced into an analysis system (e.g., HPLC), which is a benefit of the present apparatus and/or system that is not provided by a single valve component.

The automated system of the present apparatus aids analyzing the treatment solution sample. The automated system may comprise, consist essentially of, or consist of one or more of the following components: a) a substance-separation apparatus (e.g., a column), b) one or more measurement cells, c) one or more light sources, d) one or more optical filters, and/or e) a light sensor. Specifically, the present apparatus or system may comprise one or more sample measurement cells. In an illustrative example, the apparatus comprises, consists essentially of, or consists of about 1 to about 5 sample measurement cells, and at or about 1 to about 2 sample measurement cells.

The sample measurement cells of the present disclosure may comprise one or more sensors. Each sensor and/or sample measurement cell may be used to analyze the same or different families, classes, types, or individual active ingredients and chemical compounds. For example, the one or more sample measurement cells of the present apparatus may be used to analyze the same or different families, classes, types, or individual active ingredients or chemical compounds, even if more than one active ingredients or chemical compounds of the same family, class, or type are present in the treatment solution.

In an illustrative example, one particular sensor and/or sample measurement cell of the present apparatus or system (i.e., a first sample measurement cell) may be used to measure, monitor, analyze, and/or control many, multiple, or a plurality of active ingredients, such as fungicides. For example, multiple fungicides that may be analyzed by one or more sample measurement cells of the present apparatus or system include, but are not limited to imazalil, thiabendazole, fludioxonil, prochloraz, propioconazole, pyrimethanil, orthophenylphenol, azoxystrobin, difenoconazole, diphenylamine, and combinations thereof.

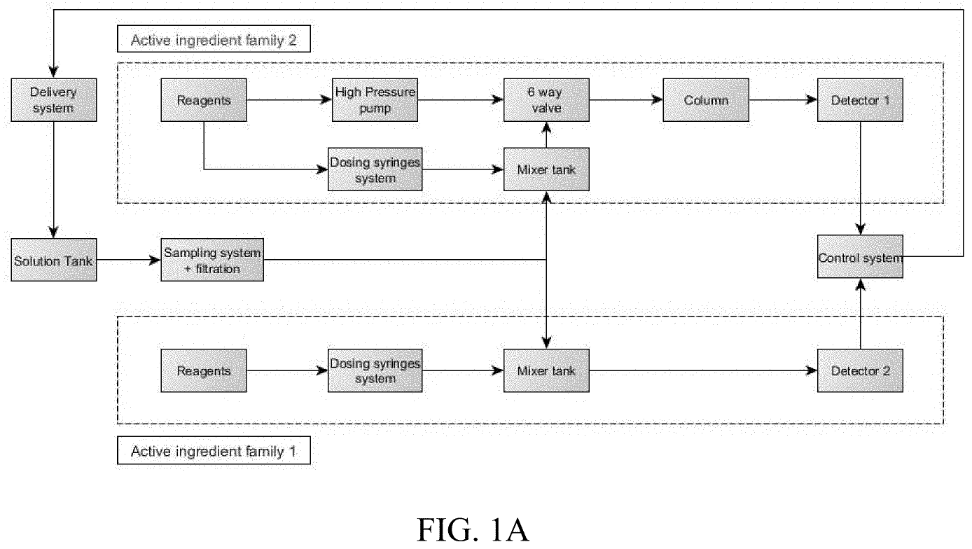

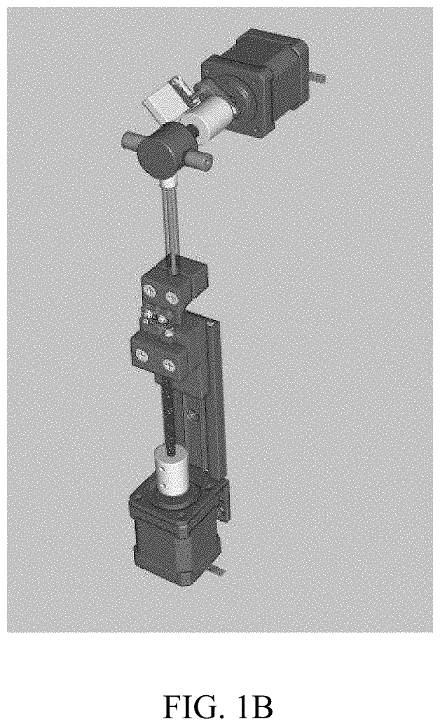

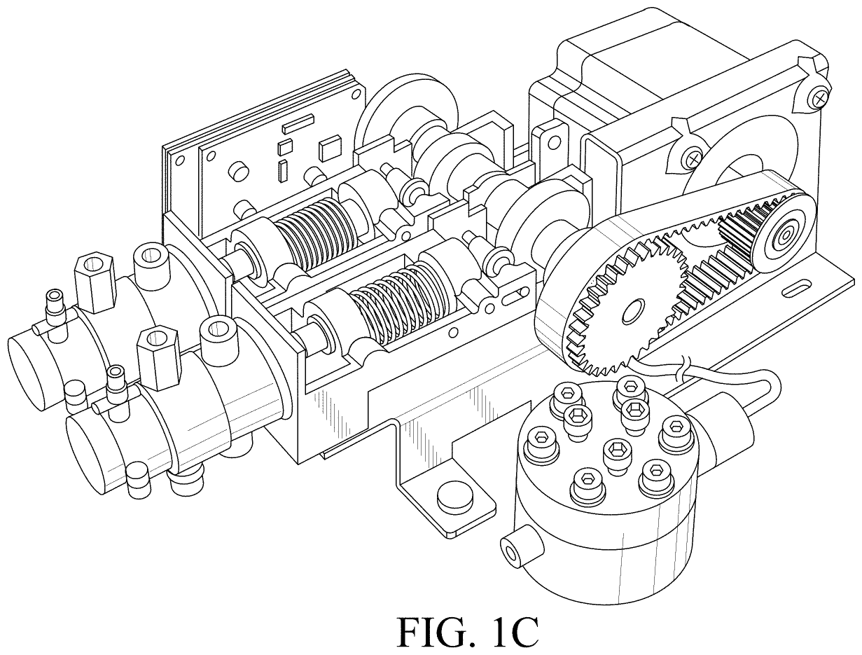

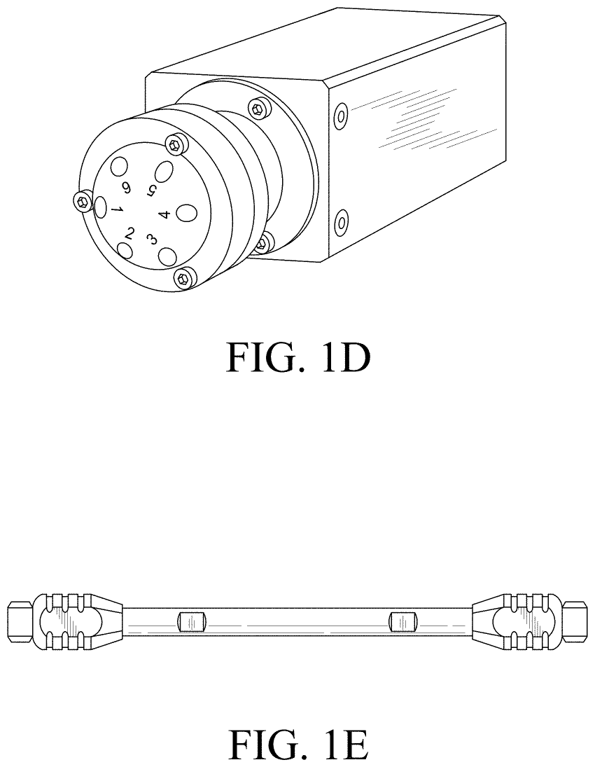

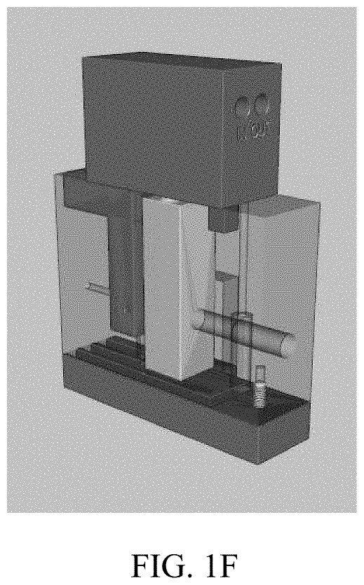

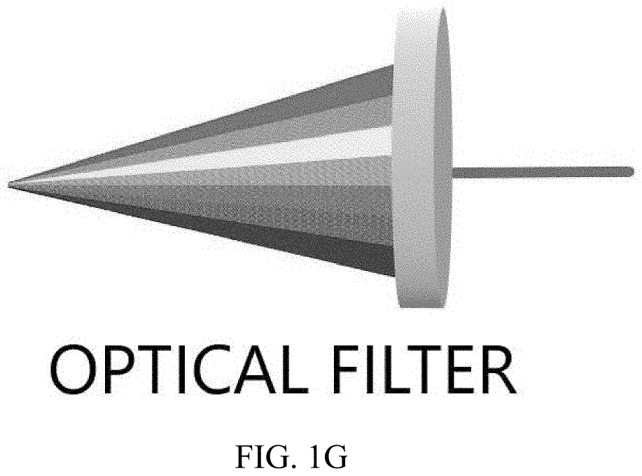

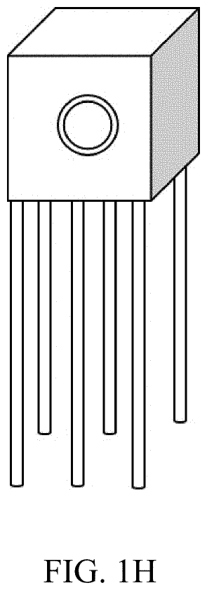

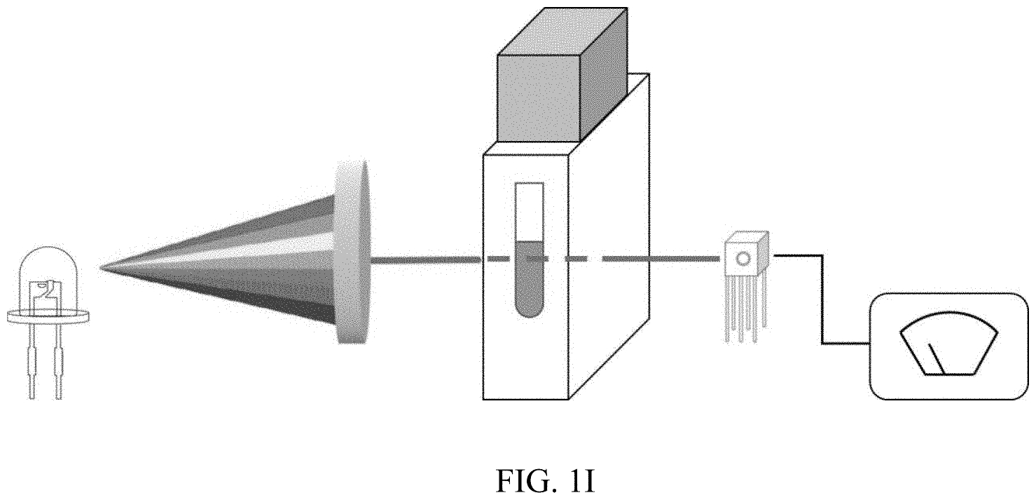

In addition to a first sample measurement cell, a second sensor and/or sample measurement cell may be comprised by the present apparatus or system of the present disclosure (see FIG. 1A). A second sensor or sample measurement cell of the present apparatus or system is available for measuring, monitoring, analyzing, and/or controlling the amount of another or a second family, class, type, or individual active ingredient, such as a sanitizing agent (see FIG. 1A). For example, a second sensor and/or sample measurement cell of the present apparatus could be used to measure, monitor, analyze, and/or control a sanitizing agent, such as peracetic acid, acetic acid, ethanol, hydrogen peroxide, chlorine, hypochlorous acid, sodium hypochlorites, calcium hypochlorites, or combinations thereof.

The present apparatus employs the one or more sample measurement cells and/or sensors as a technological tool to measure and/or detect various concentrations of one or more (e.g., a plurality) of the active ingredient and chemical compounds comprised in a treatment solution. For example, the measurement cells and/or sensors of the present apparatus may measure and/or detect various concentrations of the same active ingredient. Alternatively, the measurement cells and sensors of the present disclosure are capable of measuring and/or detecting the concentration of various active ingredients at a single or multiple time points.

The sample or modified sample may then be pumped to and/or through the apparatus to a column An illustrative column of the present apparatus is a chromatographic column. Generally, the column of the apparatus will separate the one or more active ingredients in the modified sample from each other and/or from reagents, reactants, and products in preparation for detection of the concentration of the one or more active ingredients.

The concentration of the one or more active ingredients comprised in the modified sample of treatment solution may then be assessed by one or more light sensing systems, such as one or more detectors. In one embodiment, the apparatus of the present disclosure may comprise, consist essentially of, or consist of one detector. In another embodiment, the present apparatus may comprise, consist essentially of, or consist of two detectors. In a further embodiment, the present apparatus may comprise, consist essentially of, or consist of two or more detectors (e.g., three or more). The present apparatus may be operated via a wireless connection, such as a Wi-Fi or a Bluetooth connection.

Once the electrochemical reaction between the reactants and/or reagents and the active ingredient has occurred in the modified treatment sample, the detectors of the present apparatus or system analyzes the modified treatment sample and generates a signal, such as an electrical signal. Additional components of the detector that may be necessary to help generate and/or amplify the electric signal may comprise, consist essentially of, or consist of a filter, a photosensor, and a light source. The light source of the detector may be any light source that helps generate the electrical signal at an intensity sufficient to calculate and/or ascertain the concentration value of the one or more active ingredients. An exemplary light source of the present disclosure is an LED light source.

More specifically, measuring and/or detecting a signal by the present apparatus is performed by adding the corresponding reagents to the obtained treatment solution sample resulting in chemical compounds with an absorbance peak at a specific wavelength within the range of 200 nm to 600 nm when light is applied. This absorbance peak is proportional to the concentration of the active ingredient to be measured. In order to measure absorbance of the sample, light emitted from a source passes through the sample, but part of the light is absorbed by the new chemical compounds that were formed with the addition of the reagents. The light transmitted through the sample is filtered with an optical filter that only allows a range of about 200 nm to about 600 nm wavelength of light to go through the optical filter. This light reaches a transducer that converts the light into an electrical signal that is measurable and is proportional to its intensity. This light intensity is, in turn, proportional to the concentration of the active ingredient in the sample.

Electrochemical analyses employed by the present apparatus and methods comprise, consist essentially of, or consist of combining two or more chemical compounds and/or active ingredients, and compositions or combinations thereof to generate a signal or a reading. For example, a mixture and/or mixing the reagents or reactants with the active ingredients of the present disclosure may comprise an electrochemical reaction to generate a signal. In an exemplary embodiment, the signal generated by the electrochemical reaction of the present apparatus or system is an electric signal. The electric signal generally comprises electricity, which may be detected by standard methods known in the art.

Importantly, the electric signal generated by the present apparatus or method may be correlated to and/or proportional to the amount or concentration of the active ingredient in the treatment solution. In such cases, a relationship between the signal and the concentration of the corresponding compound or active ingredients may be established. In some embodiments, a linear regression or calibration curve may be implemented to establish the relationship and/or correlation between the concentration of active ingredient present in the treatment solution and the electrical signal generated by the active ingredient.

The signal analysis system of the present apparatus comprises, consists essentially of, or consists of a computer or an electronic device. The computer or electronic device of the present disclosure may translate the electrical signal generated by a detector into a magnitude or a value. In an illustrative embodiment, the electrical signal is generated and analyzed by a control system to determine and/or identify a concentration value of the one or more active ingredients.

Typically, the control system of the present apparatus comprises, consists essentially of, or consists of a computer or electronic device that can display and/or record the data generated by the signal analysis system. In addition, the control system may compare the values of the desired and/or set point concentrations of active ingredients with the measured concentrations. Further, the control system of the present disclosure also communicates with the automated delivery system.

Thus, the apparatus and methods of the present disclosure may comprise a control system, such as a computer, to perform electronic, computational, and/or mathematical analyses to calculate, determine, and/or establish the concentration of active ingredient or chemical compounds in the treatment solution. Such electronic, computational, and/or mathematical analyses may comprise, consist essentially of, or consist of a linear regression or calibration curve and may be performed by a control system of the present apparatus. Therefore, the control system is able to read, interpret, and/or analyze the signal (e.g., electrical signal) and electronically, computationally, and/or mathematically identify the amount and/or concentration value of the one or more active ingredients in the treatment solution.

Alternatively, analysis of one or more active ingredients in one or more treatment solution samples or modified samples by the present apparatus or system comprises, consists essentially of, or consists of basic analytical chemistry methods, such as High-Performance Liquid Chromatography (HPLC) and gas chromatography (GC), as well as photometric and electrochemical techniques, and the combinations of all thereof. HPLC is a basic analytical chemistry technique used to separate, identify, quantify, and/or isolate components of a mixture or sample. Therefore, HPLC and other known sample analysis techniques may be employed to measure a concentration of the one or more active ingredients in a treatment solution sample in the present signal analysis system.

Once the amount of concentration of the one or more active ingredients in the treatment solution is determined by the control system of the present apparatus or system, the control system (e.g., a computer) further determines the concentration of active ingredient that is necessary to equilibrate the concentration of active ingredient to the desired set point concentration of the active ingredient, if any is necessary at all. Determining an additional concentration of an active ingredient that may be delivered to the treatment tank or chamber to equilibrate the concentration of active ingredient back to the set point concentration of the active ingredient is performed by the control system by subtracting the concentration of the active ingredient measured by the apparatus from the initial set point concentration of the active ingredient.

For example, in an illustrative embodiment of the present disclosure, the one or more active ingredients are not equilibrated if the concentration of the one or more active ingredients measured by the present apparatus is within about ±10%, about ±5%, about ±4%, about ±3%, about ±2.5%, about ±2%, about ±1.5%, about ±1%, or about ±0.5% of the set point concentration of the one or more active ingredients. In another embodiment, the one or more active ingredients are not equilibrated if the concentration of the one or more active ingredients is measured by the present apparatus is generally within, but is not limited to about ±10% of the set point concentration of the one or more active ingredients.

In an embodiment where the active ingredient is not equilibrated by the apparatus, the delivery pumps are not engaged at all. Instead, the active ingredient is only measured and monitored by the detectors and the control system of the apparatus, but no active ingredient formulation comprising the one or more active ingredients is delivered to the tank. Delivery of the one or more active ingredients in a treatment solution to the treatment tank may be performed manually or automatically or not at all.

Therefore, the illustrative automated delivery system may comprise, consist essentially of, or consist of a tank, a chamber, and/or a container. The tank and/or chamber of the present apparatus may contain a formulation of one or more active ingredients. In addition, the automated dosing system may further comprise one or more delivery pumps, hoses, and/or a dosifier.

For example, when the concentration of one or more active ingredients measured by the apparatus (e.g., the signal analysis system and the control system) is outside of about ±10%, about ±5%, about ±4%, about ±3%, about ±2.5%, about ±2%, about ±1.5%, about ±1%, or about ±0.5% of the desired concentration of the one or more active ingredients, one or more delivery pumps may be used to deliver an additional concentration of an active ingredient to the treatment solution in the treatment tank to equilibrate the initial concentration of active ingredient back to the set point concentration. Alternatively, the additional concentration of the active ingredient may be manually delivered to the treatment solution in the treatment tank.

Recovery of the set point concentration of the one or more active ingredients in the treatment solution comprised within the tank after delivery of the additional concentration of the active ingredient by the present apparatus provides control over the desired set point concentration in the recovered treatment solution (i.e., the treatment solution comprising added active ingredient to aid its recovery to the set point concentration). In addition, delivery and return of the concentration of the one or more active ingredients to the set point concentration in the recovered treatment solution comprised in a tank may be completely performed by the present apparatus. More specifically, the present apparatus and/or system of the present disclosure provides the capability to automatically administer, measure, monitor, maintain, deliver, and/or control the concentration of one or more active ingredients in a treatment solution comprised in a tank for treating plants and plant crops.

Once the additional concentration of the one or more active ingredients and/or treatment solution is delivered to the treatment tank, the present apparatus may perform one or more subsequent and/or final measurements to verify that the concentration of the active ingredients in the recovered treatment solution has remained or is equal to, at, or about the set point concentration of the one or more active ingredients. The present apparatus or system is also designed to record measurements conducted during the performance of the present methods.

For example, the control system of the present apparatus may electronically record and save some, all, or specific control parameters and results from some, all, or specific measurements in order to provide traceability and reproducibility necessary for administration of the same treatment protocol for plants and plant crops in the future. All such recorded measurements comprised by the apparatus may be accessed using the control system of the present disclosure. The present apparatus of the present disclosure also enables routine, continuous, automated, on-demand, and/or real-time measuring, monitoring, and/or controlling of the active ingredients of the plant crop protection treatment solutions of the present disclosure.

Moreover, the apparatus of the present disclosure may have online capabilities that are controlled by a remote system. A remote system of the present apparatus may allow or enable a user or operator to control the operations and functions of the apparatus and/or the treatment tank while not in direct contact or vicinity of the apparatus and/or treatment tank. Thus, the control system and the remote system of the present apparatus may be one and the same component or separate and different components of the present apparatus.

An illustrative control system and remote system of the present apparatus may comprise, consist essentially of, or consist of a personal computer, a tablet, a mobile or cell phone, or a laptop. Such devices may be used interchangeably as a control system and a remote system. Yet, an exemplary control system for the present apparatus is a computer or a laptop, while an exemplary remote system (i.e., a remote or remote control) for the present apparatus may comprise, consist essentially of, or consist of a device, such as a personal device.

The remote and control system may operate the present apparatus via an internet connection or a wireless internet connection, such as Wi-Fi or a Bluetooth connection. Notably, the control system and/or the remote system of the present apparatus may allow a user that is not in the same room, location, venue, and/or the same vicinity to control the operations, functions, and on/off of the present apparatus and/or treatment tanks.

The delivery system of the present apparatus comprises, consists essentially of, or consists of an automated device that receives the information or data from the control system. In this case, the control system is responsible for generating, analyzing, and/or interpreting the data indicating the amount of active ingredient formulation that should be delivered to the treatment solution in the treatment solution tank to return to the set point concentration, and communicating that information to the delivery system. The delivery system performs the transport of the additional active concentration to the treatment solution such that the concentration returns back to the set point concentration value.

### Methods of the Present Disclosure

The present disclosure is directed to methods for measuring, monitoring, and/or controlling the chemical concentration of one or more active ingredients in a solution for treating plants and plant crops, such as fruits and vegetables of any kind. More specifically, the present disclosure is related to methods of administering, monitoring, measuring, and/or controlling concentrations of compositions comprising chemical compounds and/or active ingredients that improve the shelf life and/or reduce, prevent, or delay degradation of plants and plant crops, such as fruit and/or vegetable crops.

The methods of the present disclosure may be performed manually, semi-automatically, and/or automatically. Manual methods are those methods where about 90% of the action required is performed by a person, an individual, or some external source (e.g., a robot). Semi-automatic methods are those methods where at least about 50% of the action required is performed automatically by an apparatus or device and 50% or less is performed by a person, an individual, or some external source (e.g., a robot). Finally, automatic methods are those methods where at least about 90% of the action required is performed automatically.

An automatic method of the present disclosure may be performed without any or any substantial aid from a person, an individual, or some external source (e.g., a robot). In one embodiment, an automatic method of the present disclosure is performed with the apparatus or system described herein. In another embodiment, a semi-automatic method of the present disclosure is performed with the apparatus or system described herein. Finally, a manual method may also be performed with the apparatus or system described herein.

Active ingredients of the present methods may comprise, consist essentially of, or consist of any compound or composition that is effective to improve the shelf life and/or reduce, prevent, and/or delay degradation of plants and plant crops, such as fruit and/or vegetable crops. Any effective combination of different types, kinds, and classes of active ingredients may be used individually or as a mixture or combination in the present methods. Active ingredients of the present methods comprise, consist essentially of, or consist of pesticides, insecticides, fungicides, herbicides, food additives, processing aids, sanitizing agents, benzoxaborole derivatives, ethylene inhibitors, and/or combinations thereof. Illustrative examples of the active ingredients of the present methods comprise fungicides, processing aids, and/or sanitizing agents.

An exemplary active ingredient of the present methods is a fungicide. While any fungicide may be comprised in the present methods, exemplary fungicides of the present methods comprise, consist essentially of, or consist of imazalil, thiabendazole, fludioxonil, prochloraz, propioconazole, pyrimethanil, orthophenylphenol, azoxystrobin, difenoconazole, diphenylamine, and analogs or derivatives thereof. In addition, any effective combination of fungicides may be comprised in the methods of the present disclosure.

Another illustrative example of the active ingredient of the present methods comprises, consists essentially of, or consists of a sanitizing agent. A sanitizing agent of the present disclosure comprises, consists essentially of, or consists of peracetic acid. Other sanitizing agent of the present disclosure comprises, consists essentially of, or consists of acetic acid, ethanol, hydrogen peroxide, chlorine, hypochlorous acid, sodium hypochlorites, calcium hypochlorites, or combinations thereof. Any effective combination of sanitizing agents may be comprised in the methods of the present disclosure.

Active ingredients of the present methods may be administered and/or applied to plants and plant crops to effectively extend shelf life and/or reduce, delay, or prevent degradation. Active ingredients may be used in any form, including, but not limited to, a liquid, a solid (e.g., a powder), or a gaseous composition. In particular, the present method provides application of the active ingredients or chemical compounds as a spray, a mist, a gel, a thermal and non-thermal fog, a dip, a drench, a vapor, or a gas.

An exemplary application method comprises administering the one or more active ingredients comprised within a treatment solution by spraying, dipping, and/or drenching the treatment solution onto plants and plant crops, such as fruit or vegetables crops. Additional examples of treatment administration of the one or more active ingredients of the present disclosure may include, but are not limited to, release from a sachet, a synthetic or natural film, a liner or other packaging materials, a gas-releasing generator, compressed or non-compressed gas cylinder, a droplet inside a box, or other similar methods.

Once the one or more active ingredients are released, applied, and/or administered to the plants and plant crops, the present methods are related to measuring, monitoring, and/or controlling the chemical concentration of the active ingredients in the treatment solution to ensure that the plants and plant crops are treated with an effective amount of the one or more active ingredients. The effective dose for each of the one or more active ingredients comprised in a treatment solution is entered and saved via the dosing system. Typically, the effective amount for each active ingredient in a treatment solution is dependent on the plants and plant crops to be treated, and is therefore often determined by an administrator of the present methods.

It can also be appreciated by those skilled in the art that the concentration or dose of active ingredient that is effective for one specific plant crop cannot be properly assumed to be effective for any other specific plant crop, whether those plants and plant crops are related or not. Therefore, the effective amount of each active ingredient for a specific plant crop (e.g., fruit or vegetable) must be calculated and/or experimentally determined by a user or administrator.

Once determined, the concentration of each of the one or more active ingredients to provide an effective dose to the specific plants and plant crops is entered as a set point in the dosing system of the treatment solution, which is administered in a treatment or mixing tank. Thus, the set point of each active ingredient in a particular treatment solution is the concentration of the active ingredient that provides an effective dose to the specific plant and plant crop being treated. This set point may also be the desired and/or preferred concentration of active ingredient to be maintained in the treatment solution and/or the treatment tank throughout the treatment of the plants and plant crops.

Treatment solutions comprising, consisting essentially of, or consisting of the one or more active ingredients may be applied to the plants or crops inside of a tank or a chamber. The tank may be open or closed/sealed during application of the active ingredient treatment solution. The tank or chamber may be further placed inside of a contained space (as described herein). Typically, the plants or crops, such as fruit and/or vegetable crops, are manually or robotically placed in the tank, and the tank may optionally be sealed. The treatment solution is then applied to the tank comprising the plants and plant crops at the set point concentration for a specific time period, and often at a particular temperature.

The plants and plant crops may be exposed to the treatment solution in the tank for duration of application or administration that may range from about 5 seconds to about 30 minutes, and often times less than 1 minute (e.g., 55 seconds, 50 seconds, 45 seconds, 40 seconds, 30 seconds, 20 seconds, 15 seconds, 10 seconds, and/or 5 seconds). The temperature of the tank or chamber during the treatment application may remain at a temperature, which ranges from about 1° C. to about 50° C., such as about 1° C. to about 48° C., 1° C. to about 45° C., 1° C. to about 40° C., and 1° C. to about 35° C.

During treatment of the plants and plant crops the present methods comprise obtaining a sample or an aliquot of a treatment solution (i.e., the treatment solution sample) comprised in the treatment tank. The treatment solution sample may comprise, consist essentially of, or consist of an initial concentration comprising one or more active ingredients. Once the sample of the treatment solution is obtained, the present methods may comprise preparing the treatment solution sample for analysis. For example, preparing the treatment solution sample for analysis may comprise, consist essentially of, or consist of filtering and/or diluting the sample to improve its purity and/or concentration, respectively.

Due to the presence of undesired agents (e.g., particles of dirt and other compounds) that are typically present in the original sample of a treatment solution, in some method embodiments, the sample of the treatment solution is filtered. Filtering the sample, by passing the sample through a filter (e.g., 20 μm to 50 μm filter), removes much of the undesired contaminating agents without affecting the active ingredients. Once filtered, the sample may also be diluted prior to reaching the sample measurement cell of the apparatus or system. As described below, the sample is often then mixed with corresponding reagents to produce a modified treatment solution sample. This modified sample is further analyzed.

Preparation of any treatment solution sample for analysis may comprise adding or combining one or more reactants or reagents to the sample to form a mixture. This modified treatment solution sample is then analyzed by the present methods to determine the concentration of active ingredients in order to ensure that the set point concentration is maintained. Thus, the present method comprises, consists essentially of, or consists of obtaining a treatment solution sample with an unknown, initial concentration of one or more active ingredients to be determined.

Reactants and/or reagents may be added to the sample comprising the active ingredients simultaneously or consecutively. Combining the reagents, reactants, and the treatment solution sample results in a chemical reaction (e.g., an electrochemical or chemiluminescent reaction) that generates a signal or a reading. The chemical reaction of the present method also converts the treatment solution sample into a modified treatment solution sample (“also referred to as the treatment solution sample”). The method further comprises performing analyses of the modified treatment solution sample in a measurement cell and generating a signal.

In some embodiment, a photochemical reaction of the present methods or apparatus may generate a luminescent signal, by techniques used or known in the art. However, in further embodiments, an electrochemical reaction of the present method is utilized to generate an electrical signal by adding the corresponding reagents to the obtained sample resulting in chemical compounds with an absorbance peak at a specific wavelength when light is applied. This absorbance peak is proportional to the concentration of the active ingredient to be measured.

The present methods further comprise detecting the electric signal generated by the chemical reaction that occurs in the modified treatment solution sample. After detecting the electrical signal, the electrical signal is analyzed to identify the initial concentration of the one or more active ingredients comprised in the modified treatment solution sample. Thus, the present method also comprises analyzing the electrical signal to further determine, calculate, and/or identify the initial concentration of the active ingredient in the treatment solution sample.

Importantly, the electric signal generated by the present methods may be correlated to and/or proportional to the amount or concentration of the active ingredient in the treatment solution. Thus, the present methods further comprise analyzing the electric signal generated by the modified treatment solution sample to a concentration of the one or more active ingredients in the treatment solution. The present method further comprises establishing, correlating, and/or calculating the relationship between the electric signal and the concentration of the corresponding active ingredients. In one embodiment, a linear regression or calibration curve may be utilized to analyze, establish, and/or calculate the relationship between the concentration of the active ingredients present in the treatment solution and the electrical signal generated by the active ingredients.

After the initial concentration of the one or more active ingredients has been determined and/or measured, such as according to the previously described method, the present methods may comprise comparing the measured initial concentration of the active ingredients in the treatment solution to the set point concentration of each of the one or more active ingredients. Further, the method may comprise calculating, correlating, and/or determining if the initial concentration of each of the one or more active ingredients is within a set threshold or an acceptable deviation from the set point value or concentration.

An acceptable and/or standard threshold deviation of the measured initial concentration of the one or more active ingredients in the treatment solution may be within about ±10%, about ±5%, about ±4%, about ±3%, about ±2.5%, about ±2%, about ±1.5%, about ±1%, or about ±0.5% of the set point concentration of the one or more active ingredients. If the initial concentration of the active ingredients is within the specified threshold of the set point concentration, then no further action, such as addition of supplemental active ingredient, is required. In an exemplary embodiment, if the initial concentration of the active ingredients generated by the electric signal is within the specified threshold of the set point concentration, then no further action, such as addition of supplemental active ingredient, is required. Instead, continued monitoring of the active ingredient concentration may occur to ensure the active ingredient(s) concentrations remain within the acceptable threshold of the set point concentration.

In this method embodiment, the concentration of active ingredient in the treatment solution is not equilibrated further since doing so is unnecessary. Instead, this method embodiment comprises, consists essentially of, or consists of measuring and/or monitoring the active ingredient concentrations with no delivery of an additional concentration of the one or more active ingredients back into the tank of a treatment solution. Alternatively, if the initial concentration of any of the one or more active ingredients are outside of the acceptable threshold of the set point concentrations (as may be specified by a user or administrator), then additional action, such as controlling and/or equilibrating the concentrations of the one or more active ingredients, may be required.

In one embodiment of the present methods, controlling and/or equilibrating a desired concentration of the one or more active ingredients to a set point concentration in a treatment solution is required. Thus, a method of controlling and/or equilibrating the concentration of one or more active ingredients in a treatment solution of the present disclosure may comprise, consist essentially of, or consist of measuring the initial concentration of the one or more active ingredients, such as according to the method previously described. In addition, the present methods may comprise assessing the concentrations of the one or more active ingredients to determine when the concentrations are outside of an acceptable threshold of deviation from the desired or set point concentration of each of the one or more active ingredients. Assessment of the initial concentration may comprise calculating, correlating, and/or determining if the measured initial concentration of each of the one or more active ingredients is within a set threshold or acceptable deviation from the set point value or concentration.

For example, when the concentration of the one or more active ingredients measured is outside of about ±10%, about ±5%, about ±4%, about ±3%, about ±2.5%, about ±2%, about ±1.5%, about ±1%, or about ±0.5% of the set point concentration of the one or more active ingredients, the present methods comprise, consist essentially of, or consist of delivery of an addition concentration of an active ingredient, a treatment solution, or a carrier to the treatment tank comprising the treatment solution to equilibrate the initial concentration of active ingredient back to the set point concentration. In some embodiments, the methods comprise delivering a carrier comprising an additional concentration of the one or more active ingredients to the treatment solution to correct any deviation from the acceptable threshold concentration of the one or more active ingredients. For example, delivery of additional active ingredient to the treatment solution will increase the concentration of the active ingredient to the set point and/or desired concentration. Alternatively, delivery of the carrier without any additional active ingredient, will dilute the amount of active ingredient in the treatment solution such that the amount of active ingredient will decrease to the set point and/or desired concentration.

Delivery of the additional concentration of the one or more active ingredients or the carrier to a treatment solution in the treatment tank may be performed manually, automatically, or not at all. In one embodiment, the apparatus or equipment used in the present methods may comprise an alarm and/or shutoff mechanism that prevents overdosing and shuts down the apparatus should a malfunction occur. In another embodiment, the additional concentration of the one or more active ingredients is at or about an amount equal to the difference between the set point concentration of the one or more active ingredients and the initial and/or second concentration of the one or more active ingredients.

Thus, the method further comprises recovering the concentration of the one or more active ingredients to the set point concentration after delivery of the additional concentration of active ingredients. The methods of delivering the additional concentration of active ingredients and thus, recovering the initial concentration of active ingredients to the set point of the one or more active ingredients in the treatment solution also enables the method of controlling and/or equilibrating the set point concentration in the recovered treatment solution (i.e., the treatment solution comprising added active ingredient to aid its recovery to the set point concentration).

Accordingly, the method of controlling and/or equilibrating the set point concentration in the treatment solution may comprise, consist essentially of, or consist of recovering the initial concentration of active ingredients to the set point of the one or more active ingredients in the treatment solution. Thus, the methods of the present disclosure comprise administering, measuring, monitoring, maintaining, delivering, recovering, and/or controlling the set point concentration of one or more active ingredients in a treatment solution comprised in a tank for treating plants and plant crops.

Once the additional concentration of the one or more active ingredients, carrier, and/or treatment solution is delivered to the treatment tank in order to recover the concentration back to the set point concentration, the present methods comprise performing one or more subsequent or final measurements of the concentration of the one or more active ingredients in the recovered treatment solution to verify that it remains at or about the desired concentration (e.g., the set point concentration) of the one or more active ingredients or within the threshold. Thus, the present methods further comprising measuring and/or verifying the concentration of the one or more active ingredients after delivery and/or recovery to ensure that the concentration of the active ingredients comprised in the recovered treatment solution is equal to, at, or about the set point concentration of the one or more active ingredients.

In doing so, the present methods may further comprise recording and/or saving active ingredient concentration measurements obtained throughout the methods. For example, the present methods may comprise electronically recording and/or saving some, all, or specific control parameters, concentration measurements, and/or results in order to provide traceability and reproducibility necessary for administration of the same treatment protocol for the same of different plants and plant crops in the future. Notably, the present method further comprises accessing the recorded and/or saved measurements using a control system or a remote system of the present disclosure.

### EXAMPLES

Illustrative embodiments of the methods of the present disclosure are provided herein by way of examples. While the concepts and technology of the present disclosure are susceptible to broad application, various modifications, and alternative forms, specific embodiments will be described here in detail. It should be understood, however, that there is no intent to limit the concepts of the present disclosure to the particular forms disclosed, but on the contrary, the intention is to cover all modifications, equivalents, and alternatives consistent with the present disclosure and the appended claims. The following experiments were conducted to exemplify various aspects, features, and characteristics of the methods and apparatus described herein that are believed to provide unexpected results and technical benefits over the current technologies regarding the same.

### Example 1

### An Embodiment of the Apparatus of the Present Disclosure

FIG. 1A is a schematic that denotes the various components of the present apparatus. As described in detail herein, the present methods or apparatus comprise, consist essentially of, or consist of one or more of the following components: 1) a sampling and filtration system, such as one or more sample measurement cells, 2) a pump (e.g., a high pressure pump), 3) a valve (e.g., an injection or 6-way valve), 4) a column (e.g., a chromatographic column), 5) one or more light sensing systems (e.g., detectors 1 and 2), 6) one or more dosing syringes systems, 7) one or more treatment and/or a mixer tanks, 8) a delivery system, 9) a solution tank, 10) a control system, and 11) reactants and/or reagents.

In addition, FIGS. 1B-1I provide schematics of illustrative embodiments of one or more of each of the apparatus components mentioned in FIG. 1A. More specifically, images are shown of one prototypic embodiment of components of the present apparatus including, but not limited to: 1) a dosing system (FIG. 1B), 2) a high-pressure pump (FIG. 1C), 3) a 6-way valve (FIG. 1D), 4) a chromatographic column (FIG. 1E). Further, FIGS. 1F-1I provide schematics of illustrative embodiments of one or more of each of the components of the one or more light sensing systems (e.g., light sensing system 1 and 2) including, but not limited to: 1) a light source, such as an LED light source (FIG. 1F), 2) a filter (FIG. 1G), and 3) a photo sensor (FIG. 1H). Finally, FIG. 1I shows a schematic of how the one or more electric signals, generated by and correlated to the concentration of the one or more active ingredients, is directed, amplified, and/or detected by the one or more light sensing systems.

### Example 2

### Detection of a Concentration of Multiple Fungicide Active Ingredients

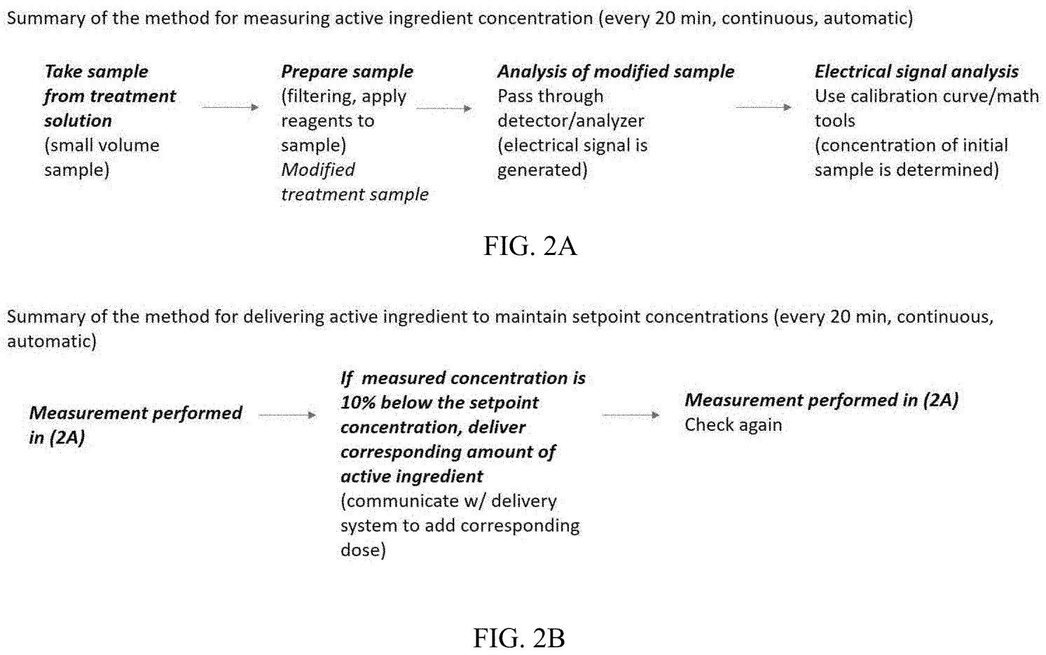

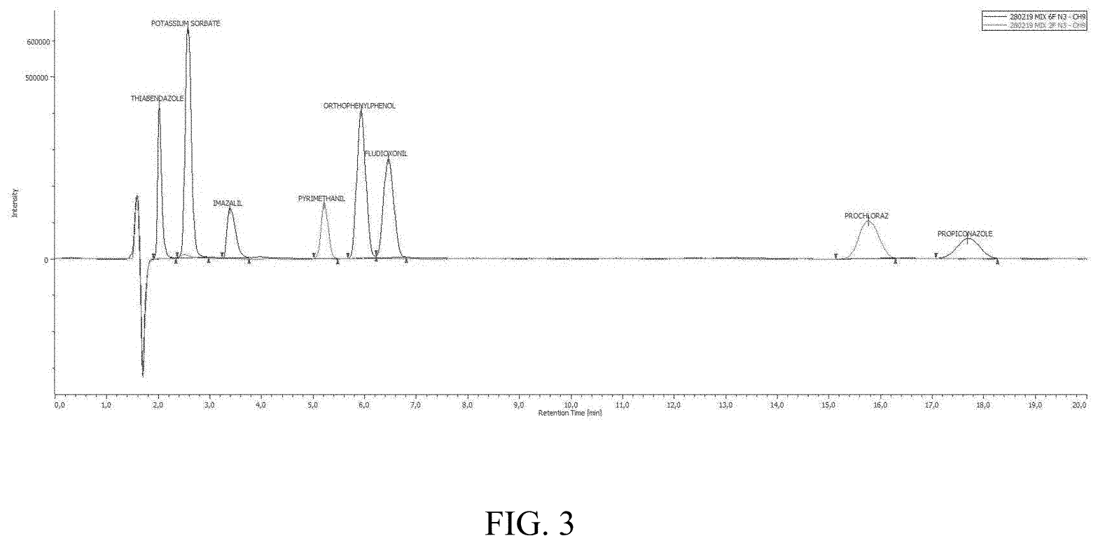

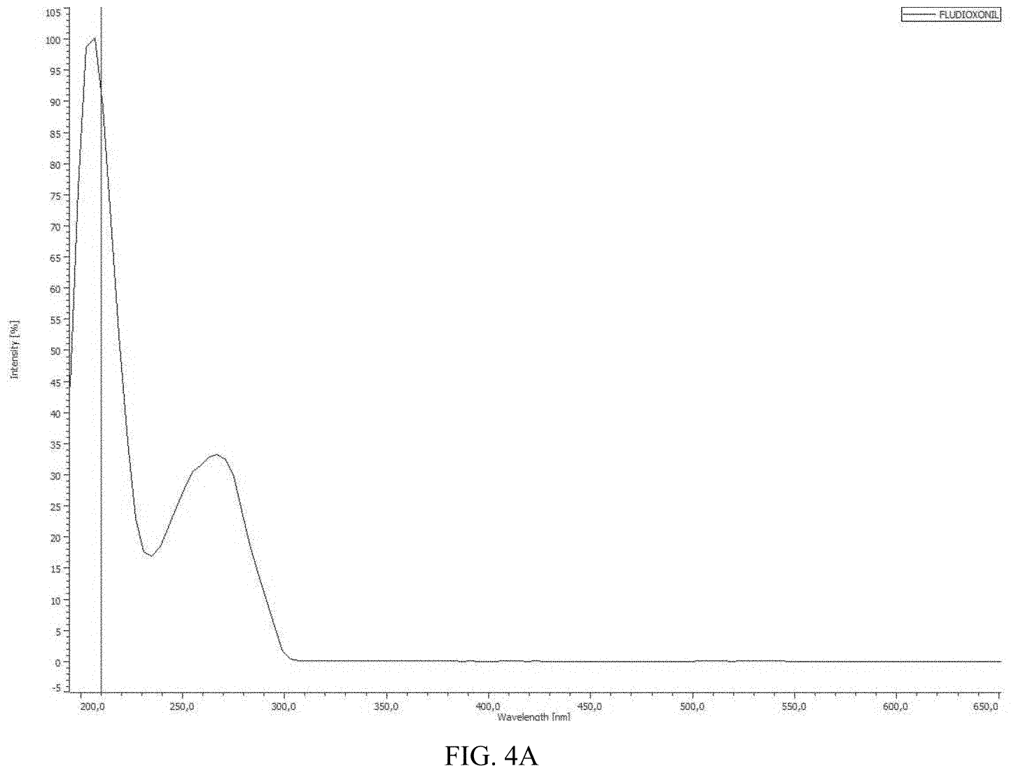

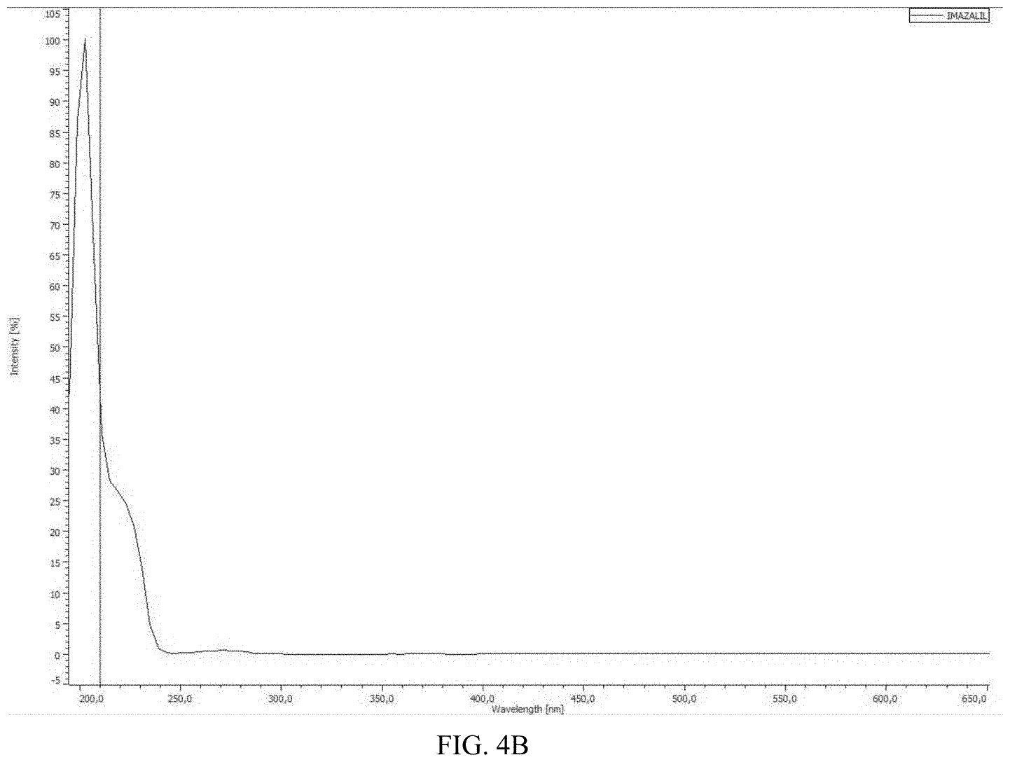

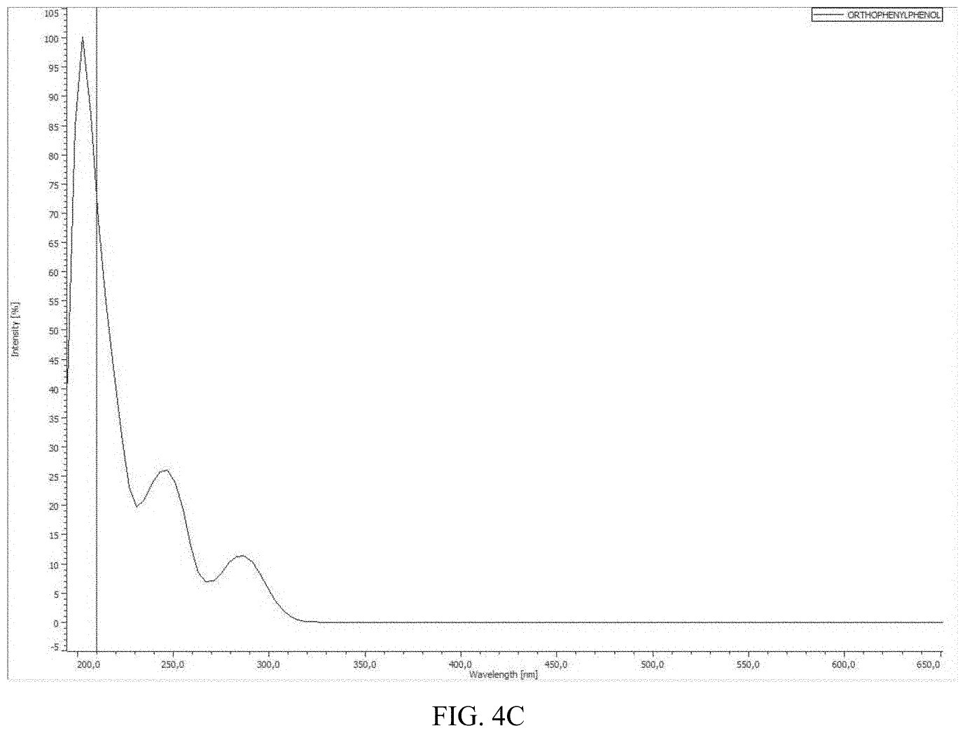

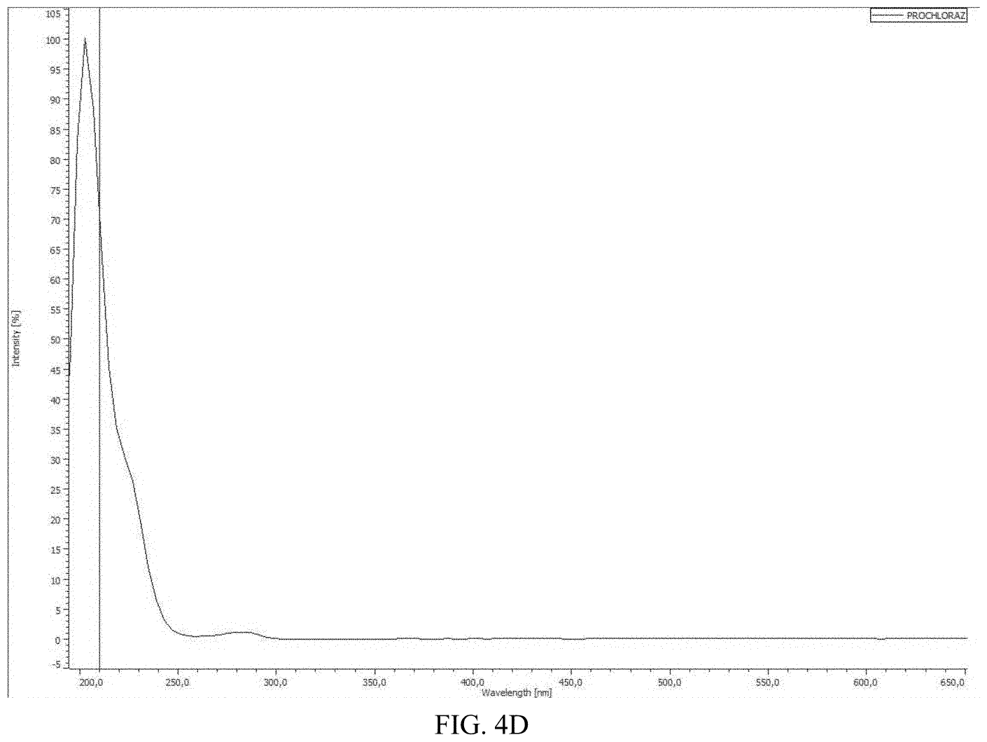

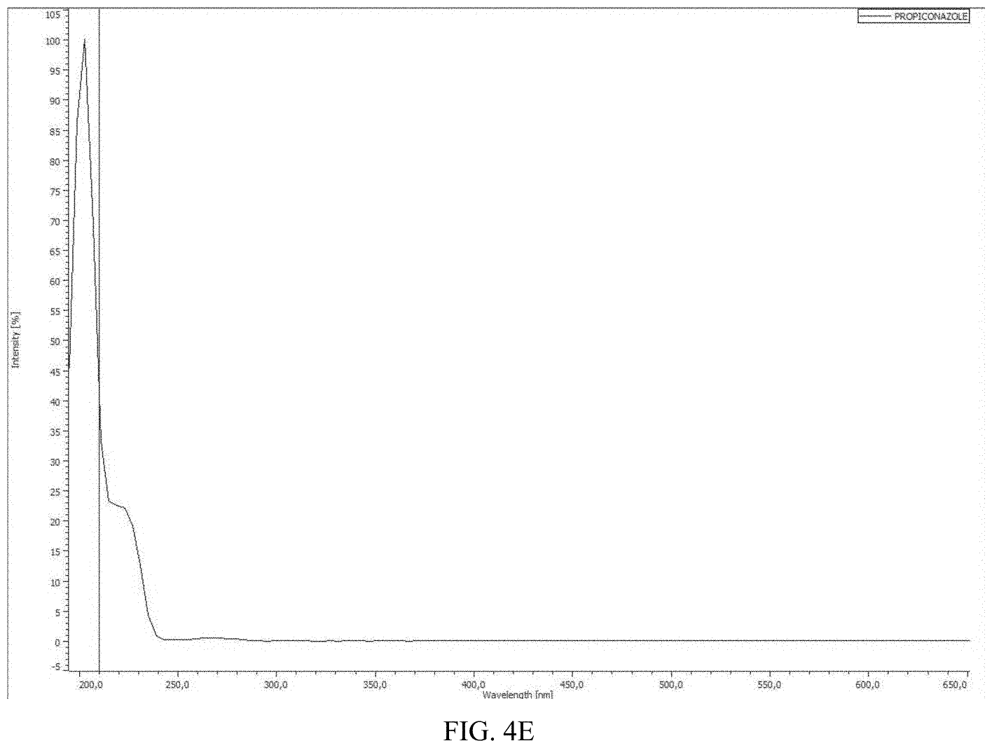

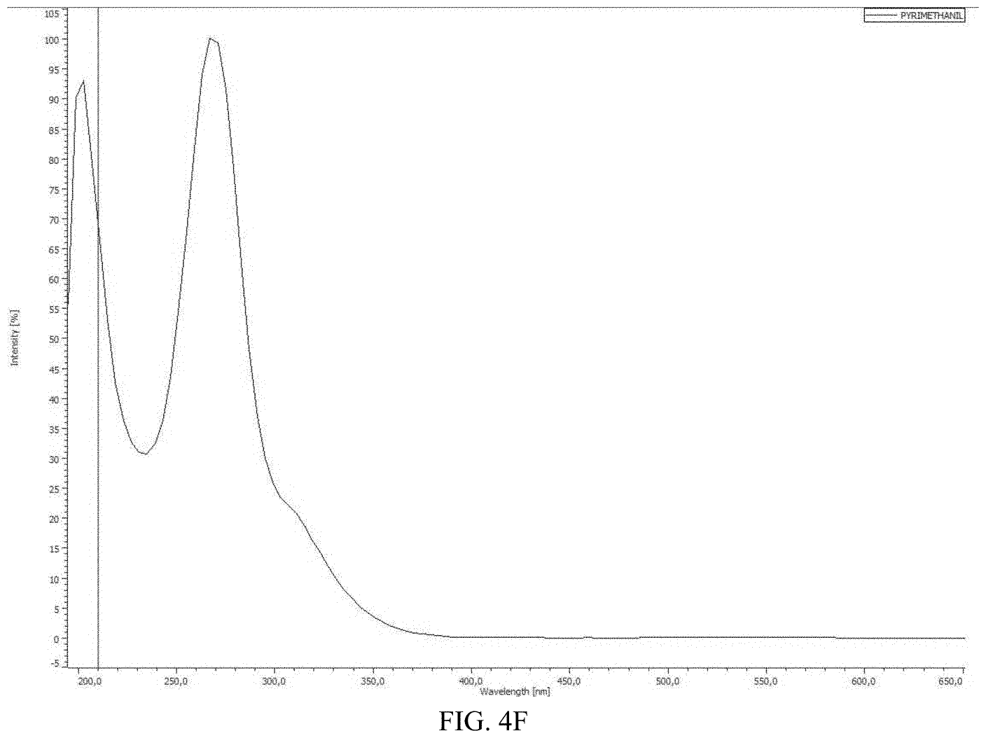

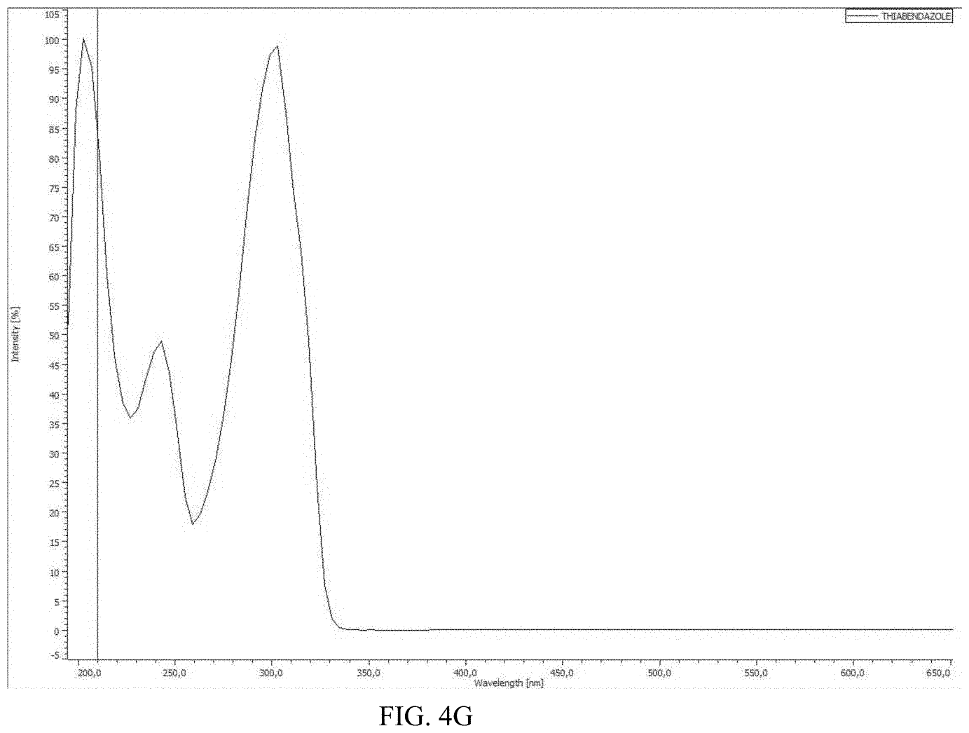

This example is related to demonstrating the ability of the present methods and apparatus to accurately detect the presence of active ingredients, such as fungicides, in a treatment solution. FIGS. 2A and 2B are schematics that represent the basic process steps and flow for the present methods. For example, FIG. 3 demonstrates that multiple different fungicides were detected in a single treatment solution. This example further demonstrates that each of the active fungicides were able to be separated and individually detected by HPLC. More specifically, the present methods and apparatus were able to separate and clearly detect the concentration of these active ingredients: fludioxonil (FIG. 4A), imazalil (FIG. 4B), orthophenylphenol (FIG. 4C), prochloraz (FIG. 4D), propioconazole (FIG. 4E), pyrimethanil (FIG. 4F), and thiabendazole (FIG. 4G).

Therefore, this example demonstrates that each of the active fungicides comprised in a single treatment solution were able to be individually separated, detected (e.g., by HPLC), and further quantitated using a calibration curve to determine one or more of their initial, second, third, or final concentrations.

### Example 3

### Detection of a Concentration of a Sanitizing Agent Active Ingredient

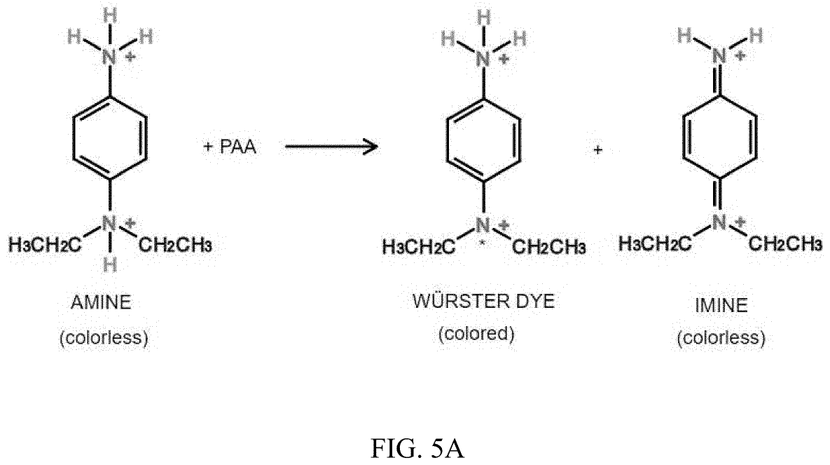

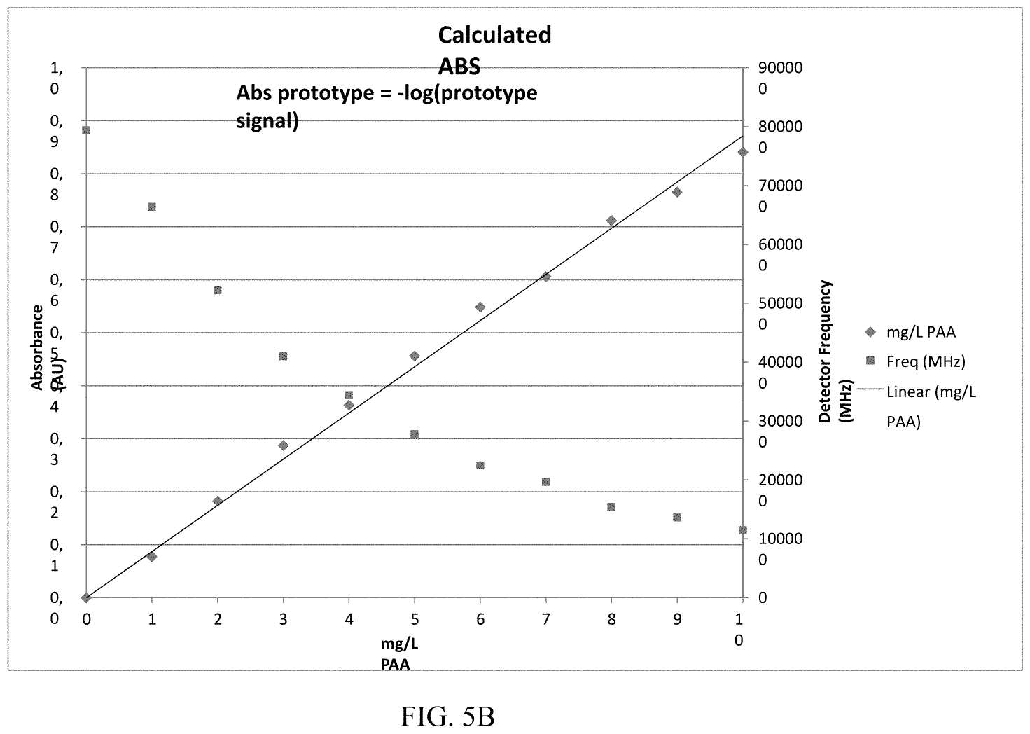

This example relates to demonstrating the ability of the present methods and apparatus to accurately detect the presence of active ingredients, such as sanitizing agents, in a treatment solution. In this example, peracetic acid is the illustrative sanitizing agent. For example, FIG. 5A demonstrates the chemical reaction that occurs in order for peracetic acid (PAA) to be detected by the present methods and apparatus. An electric signal is generated after the chemical reaction of FIG. 5A occurs.

That electric signal is then detected by the present methods and apparatus. Once detected, the electric signal is correlated to a specific concentration of the PAA FIG. 5B). The specific concentration of PAA correlating with the electric signal (FIG. 5B) is then compared to the set point value of the concentration of PAA. If the specific concentration of PAA is outside of a ±10% threshold of the set point concentration of PAA, a third or final concentration of PAA will be prepared. The third or final concentration of PAA is at or about an amount equal to the difference between the set point concentration of the PAA and the detected concentration of the PAA.

### Example 4

### Equilibration of a Fludioxonil Solution for Fruit Treatment in Tanks

This experiment was conducted to test the effect of the present methods and apparatus on a treatment solution comprising a concentration of fludioxonil, a fungicide. In this embodiment of the present method, pome fruits were treated with a crop treatment solution comprising 460 mg/L of active ingredient. The concentration of the active ingredient in the treatment solution comprised in a tank was observed to decrease over time.

Since fruit are constantly and/or continuously entering and exiting the mixture tank, which often comprises water (i.e., as a carrier), it is necessary to measure the concentration of the active ingredient continuously and/or to deliver the corresponding amount to maintain a constant concentration according to the set point concentration. For example, the set point of the present apparatus embodiment is 460 mg/L of fludioxonil.

When the concentration of fludioxonil fell to 400 mg/L of fludioxonil (i.e., about −13%) after 10 Ton of fruit were treated, the present apparatus or system of the present disclosure is able to detect the lower or reduced concentration. In response, the present apparatus automatically replenishes the 60 mg/L amount of fludioxonil to bring it back up to 460 mg/L set point within a very short time, such as only a few seconds (e.g., 3-5 seconds). A final measurement is performed by the present apparatus to confirm that the concentration of the fludioxonil was equilibrated back to 460 mg/L.

### Example 5

### An Embodiment of the Methods of the Present Disclosure

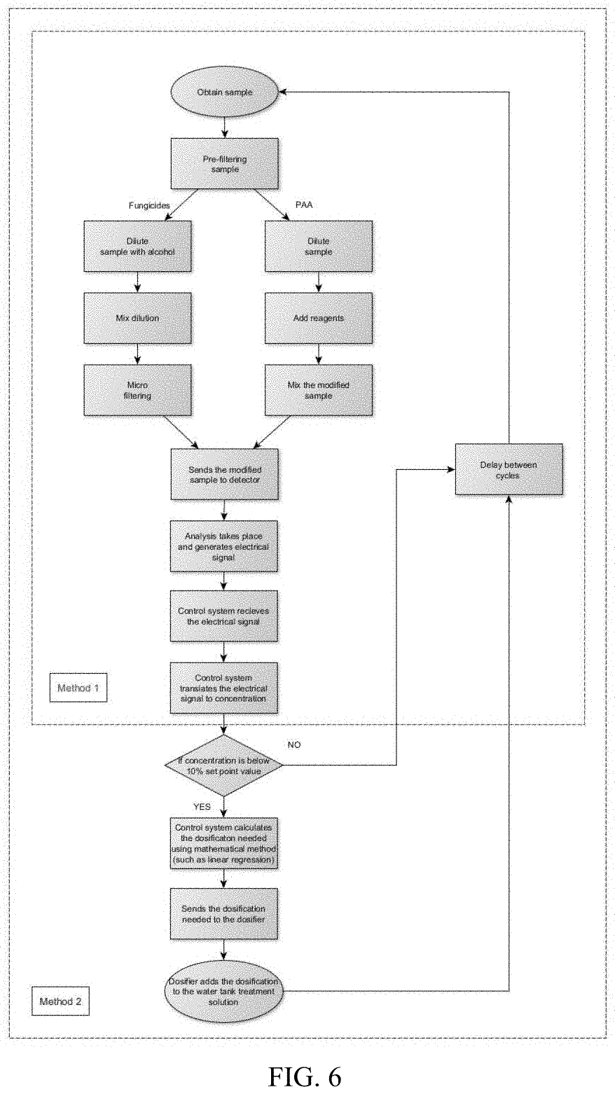

A final flow chart embodiment of the present method of measuring and monitoring a set point concentration of one or more active ingredients is shown in FIG. 6. The present method of measuring and monitoring comprises, consists essentially of, or consists of obtaining a sample of the treatment solution in the tank comprising an initial concentration of the one or more active ingredients (see FIG. 6). This sample of the treatment solution typically contains active ingredients, as well as particles of dirt and other compounds. These contaminants may affect consistency of the sample analysis.

Therefore, sample preparation prior to analysis may occur due to the need to remove the presence of undesired agents in the original sample (e.g., dirt and other contaminants) In some embodiments, the treatment solution will pass through a filter to remove as much of the contaminating agents as possible without affecting the quality and/or concentration of the active ingredients. Once filtered, the sample reaches the one or more measurement cells where the one or more active ingredients may be diluted. Samples may be diluted with water, saline, and/or alcohol (see FIG. 6). In addition, reagents may be added to the filtered and/or diluted treatment solution sample, so that the resulting modified treatment solution sample may be analyzed (see FIG. 6).

The modified treatment solution sample is typically analyzed in the one or more measurement cells. Corresponding measurement techniques may be applied to determine concentration of the sample. These measurement techniques typically comprise and/or generate electrical signals that provide “a reading” (see FIG. 6). The electric signals are typically generated, measured, and interpreted by control systems to determine the initial concentration value of the one or more active ingredients after any filtering and/or diluting (see FIG. 6).

The initial concentration value is compared to the set point concentration value (see FIG. 6). If the initial concentration, as measured and/or interpreted, falls within the allowed deviation of the set point concentration (i.e., at or about ±10%), then the delivery mechanism of the claimed apparatus or system will not engage, and no volume or additional concentration of the one or more active ingredients will be added. Nonetheless, a new sample may be obtained from the treatment solution, which may be measured and monitored, once, repeatedly, and/or continuously, as described herein.

In addition to the methods of measuring and monitoring the concentration of one or more active ingredients, if the initial concentration of the one or more active ingredients, as measured and/or interpreted, exceeds or falls below the allowed deviation from the set point value (i.e., at or about ±10%), an additional concentration of the corresponding amount or concentration (volume) of the one or more active ingredient formulations may be delivered to the treatment solution tank (see FIG. 6). This delivery may occur manually, such as by a user or an operator, or automatically, such as by a piece of equipment or an instrument, such as a dosifying system. In an illustrative embodiment, the dosifying system comprises an injection syringe and/or valve system and enables the extraction and delivery of particular doses and/or concentrations of active ingredients to or from a treatment solution (see FIG. 6).

A subsequent or final measurement of the concentration of the one or more active ingredients in the treatment solution of a tank is obtained. Ideally, the concentration measured is within the allowed deviation of the set point value, and no additional delivery is necessary. Instead, the treatment solution of the tank will be continuously measured and monitored until additional readings indicate that the concentration of one or more active ingredients is outside of the acceptable deviation, in which case equilibration, delivery, recovery, and control will ensue.

The preceding description enables others skilled in the art to utilize the technology in various embodiments and with various modifications as are suited to the particular use contemplated. In accordance with the provisions of the patent statutes, the principles and modes of operation of this apparatus and method have been explained and illustrated in exemplary embodiments. Accordingly, the present disclosure is not limited to the particular embodiments described and/or exemplified herein.

It is intended that the scope of the present technology be defined by the following claims. However, it must be understood that this apparatus and method may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

The scope of this apparatus and method should be determined, not only with reference to the above description, but should instead be determined with reference to the appended claims, along with the full scope of equivalents to which such claims are entitled. It is anticipated and intended that future developments will occur in the arts discussed herein, and that the disclosed compositions and methods will be incorporated into such future examples.

Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the apparatus and method and that the technology within the scope of these claims and their equivalents be covered thereby. In sum, it should be understood that the apparatus and method are capable of modification and variation and is limited only by the following claims.

What is claimed is:

1. A method for measuring and monitoring the concentration of one or more active ingredients in a treatment solution, the method comprising:
a) obtaining a treatment solution sample with an initial concentration comprising one or more active ingredients,
b) preparing the treatment solution sample for analyzing the concentration of the one or more active ingredients resulting in a modified treatment solution sample,
c) performing analysis of the modified treatment solution sample in a measurement cell and generating an electrical signal, and
d) analyzing the electric signal to identify the initial concentration of the one or more active ingredients comprised in the modified treatment solution sample.

2. The method of claim 1, wherein the method is performed in real-time, continuously, or on-demand by an automated system.

3. The method of claim 1, wherein a regression calibration curve is used to analyze the electric signal to identify the initial concentration of the one or more active ingredients.

4. The method of claim 1, wherein the one or more active ingredients are selected from the group consisting of pesticides, insecticides, fungicides, herbicides, food additives, processing aids, sanitizing agents, benzoxaborole derivatives, and ethylene inhibitors.

5. The method of claim 1, wherein the one or more active ingredients are a fungicide.

6. The method of claim 5, wherein fungicide is selected from the group consisting of imazalil, thiabendazole, fludioxonil, prochloraz, propioconazole, pyrimethanil, orthophenylphenol, azoxystrobin, difenoconazole, and diphenylamine.

7. The method of claim 1, wherein the one or more active ingredients are a sanitizing agent.

8. The method of claim 7, wherein the sanitizing agent is peracetic acid, acetic acid, ethanol, hydrogen peroxide, chlorine, hypochlorous acid, sodium hypochlorites, calcium hypochlorites, or combinations thereof.

9. A method for controlling a desired concentration of one or more active ingredients in a treatment solution, the method comprising:
a) measuring the initial concentration of the one or more active ingredients according to the method of claim 1,
b) assessing the measured initial concentration of the one or more active ingredients in the treatment solution to determine when it is outside the acceptable threshold of deviation from the desired concentration of the one or more active ingredients,
c) delivering a carrier or an additional concentration of the one or more active ingredients to the treatment solution to correct any deviation from the acceptable threshold, and
d) performing a subsequent measurement of the concentration of the one or more active ingredients in the treatment solution to confirm that it remains at or about the desired concentration of the one or more active ingredients or within the threshold.

10. The method of claim 9, wherein delivering is performed manually or automatically.

11. An apparatus or an integrated system that automatically measures, monitors, and optionally controls the concentration of one or more active ingredients in a treatment solution.

12. The apparatus of claim 11, further comprising an automated sample preparation system.

13. The apparatus of claim 11, further comprising an automated system for analyzing the sample.

14. The apparatus of claim 11, further comprising a signal analysis system.

15. The apparatus of claim 11, further comprising a control system.

16. The apparatus of claim 15, wherein the control system comprises a plurality of recorded measurements of the one or more active ingredient concentrations.

17. The apparatus of claim 11, further comprising an automated delivery system.

18. The apparatus of claim 11, further comprising a control system and an automated delivery system.

19. The apparatus of claim 18, wherein the control system optionally communicates with the automated delivery system to perform delivery of the corresponding amount of the one or more active ingredients or a carrier when the measured concentration is outside the acceptable threshold of deviation from the desired concentration of the one or more active ingredients.

20. The apparatus of claim 11, further comprising one or more sensors selected from the group consisting of thermometers, hygrometers, and flow meters.
